(19) United States
(12) Patent Application Publication
(10) Pub. No.: US 2011/0160591 A1
(43) Pub. Date: Jun. 30, 2011

(54) FETAL HEART RATE MONITOR WITH WIDE SEARCH AREA

(75) Inventors: Lowell Smith, Niskayuna, NY (US); Mirsaid Seyed-Bolorforosh, Guilderland, NY (US); Kai Thomenius, Clifton Park, NY (US); David Mills, Niskayuna, NY (US); Scott D. Cogan, Clifton Park, NY (US); Ashit Pandit, Columbia, MD (US); Srikanth Muthya, Laurel, MD (US)

(73) Assignee: GENERAL ELECTRIC COMPANY, Schenectady, NY (US)

(21) Appl. No.: 12/649,735

(22) Filed: Dec. 30, 2009

(51) Int. Cl. A61B 8/00

(52) U.S. Cl. 600/453

(57) ABSTRACT

A continuous, non-invasive fetal heart rate measurement is produced using an ultrasound probe positioned on the abdomen of the mother. The ultrasound probe includes a plurality of ultrasound transducers that are positioned within a housing having a transmission surface. The transmission surface is configured to defocus the individual ultrasound beams created by the plurality of ultrasound transducers. The transmission surface defocuses the ultrasound beam and creates a wider area of coverage for the ultrasound probe. The controller contained within the heart rate monitor selectively activates different combinations of the plurality of ultrasound transducers to reduce the signal-to-noise ratio while allowing the ultrasound probe to locate the fetal heart beat and subsequently increase the signal-to-noise ratio during continuous heart rate monitoring.

### BACKGROUND OF THE INVENTION

The present disclosure generally relates to a method and apparatus for determining the heart rate of a subject. More specifically, the present disclosure particularly relates to a method and apparatus for determining the beat-to-beat heart rate of a fetus.

Fetal monitoring (i.e., monitoring of the fetal condition during gestation and during labor and delivery) usually comprises monitoring uterine activity and the fetal beat-to-beat heart rate. The fetal heart rate, which provides an indication of whether the fetus is sufficiently supplied with oxygen, is preferably calculated from beat to beat.

To obtain a signal indicative of the fetal heart rate prior to rupture of the membranes, a noninvasive monitoring technique must be used. The most widely adopted measurement technique involves measuring the Doppler shift of an ultrasound signal reflected by the moving fetal heart.

In accordance with a known ultrasonic detection technique, an ultrasound transducer or transducer array is placed externally on the pregnant woman's abdomen and oriented such that the transmitted ultrasound waves impinge upon the fetal heart. The reflected ultrasound waves are received either by the same or by a different ultrasound transducer or transducer array. The Doppler shift of the reflected ultrasound wave is directly related to the speed of the moving parts of the heart, e.g., the heart valves and the heart walls.

Although the Doppler ultrasound is a widely accepted method of monitoring fetal heart rate, ultrasound fetal heart rate monitoring has several drawbacks. One of these drawbacks is that current ultrasound fetal heart rate monitors are only able to listen for a fetal heart rate within a limited volume, focused directly underneath the ultrasound transducer probe. If the fetus moves outside of this ultrasound sampled volume, the fetal heart rate signal can be lost completely, resulting in the need for a clinician or nurse to adjust the position of the ultrasound probe to find the lost fetal heart signal.

### BRIEF DESCRIPTION OF THE INVENTION

The present disclosure relates to a method and apparatus for determining the beat-to-beat heart rate of a fetus. In a disclosed embodiment, the continuous, non-invasive fetal heart rate measurement is produced using a plurality of ultrasonic transducers contained within an ultrasound probe attached to the abdomen of a pregnant patient. One or more ultrasound transducers generate an ultrasound signal or beam that is reflected by the fetal heart and received by one or more of the ultrasound transducers. Based upon the received signal, the fetal heart rate monitor generates the heart rate of the fetus.

The fetal heart rate monitor of the present disclosure includes an ultrasound probe that is positioned on the abdomen of the patient. In one embodiment of the disclosure, the ultrasound probe includes a plurality of individual ultrasound transducers that are each operable to generate an ultrasound beam from the probe housing. In one embodiment, the ultrasound probe includes nine ultrasound transducers.

The ultrasound probe of the present disclosure is formed with a transmission surface that is coupled to or formed on the housing of the probe. Each of the ultrasound transducers are positioned such that the ultrasound beam generated by each of the transducers travels through the transmission surface. In one embodiment, the ultrasound transducers are mounted to the back surface of the transmission surface.

The transmission surface is configured to defocus the ultrasound beam coming from the housing. In an embodiment including nine ultrasound transducers, the transmission surface is created such that the beam axes of eight outer transducers diverge away from the center axis of a center transducer. The use of the transmission surface to defocus the ultrasound beams from each of the plurality of ultrasound transducers increases the effective area of coverage of the ultrasound probe.

The disclosure is further directed to a method of operating a fetal heart rate monitor that includes an ultrasound probe having the plurality of ultrasound transducers. The fetal heart rate monitor can transmit and receive signals from multiple combinations of the ultrasound transducers, where each combination may include less than all of the plurality of ultrasound transducers. A controller for the fetal heart rate monitor may activate each combination of the plurality of ultrasound transducers. After each combination has been activated, the controller may determine which of the combinations of ultrasound transducers detects the heart beat. Once the controller determines which of the combinations detects the heart beat, the controller operates only the determined combination to monitor the fetal heart rate.

If more than one combination of the ultrasound transducers detects the heart beat, the system determines which combination is most effective at sensing the heart beat. Based upon this selection, the controller operates only the selected combination to monitor the heart rate of the fetus. If the heart beat is lost during monitoring, such as due to movement of the fetus, the controller again activates all of the combinations of the transducers to determine which combination detects the heart beat. If none of the combinations detects a heart beat, the system directs an operator to move the ultrasound probe on the abdomen of the patient.

Various other features, objects and advantages of the invention will be made apparent from the following description taken together with the drawings.

### BRIEF DESCRIPTION OF THE DRAWINGS

The drawings illustrate the best mode presently contemplated of carrying out the disclosure. In the drawings:

FIG. 1 depicts a pregnant patient utilizing a fetal heart rate monitor;

FIG. 2 is a schematic illustration of the ultrasound probe utilized in accordance with one embodiment of the present disclosure;

FIG. 3 is an illustration of the ultrasound sampled volume of a prior art ultrasound probe having a traditional, flat transmission surface;

FIG. 4 is a first embodiment of the transmission surface of the ultrasound probe in accordance with the present disclosure;

FIG. 5 is a second embodiment of the transmission surface of the ultrasound probe of the present disclosure;

FIG. 6 is an illustration of the expanded ultrasound volume utilizing the defocused ultrasound probe of the present disclosure;

FIG. 7 is a graphic depiction of the enhanced viewing range of the ultrasound probe having a defocusing transmission surface;

FIG. 8a is an illustration of the ultrasound volume and signal-to-noise ratio when activating all of the ultrasound transducers;

FIG. 8b is a schematic illustration of the ultrasound volume and signal-to-noise ratio when activating only a grouping of the ultrasound transducers;

FIG. 8c is a graphic illustration of the ultrasound volume and signal-to-noise ratio when activating only a single ultrasound transducer;

FIG. 9a is a graphic illustration showing the ultrasound beam from a first grouping of the ultrasound transducers;

FIG. 9b is an illustration of the ultrasound beam from a second of the ultrasound transducers;

FIG. 10 is a graphic depiction of one embodiment of the ultrasound transducer grouping;

FIG. 11 is a flowchart depicting the steps utilized to search for a monitor a fetal heart rate using less than all of the ultrasound transducers; and

FIG. 12 is a schematic illustration of the operating components of the operating components of the fetal heart rate monitor.

### DETAILED DESCRIPTION OF THE INVENTION

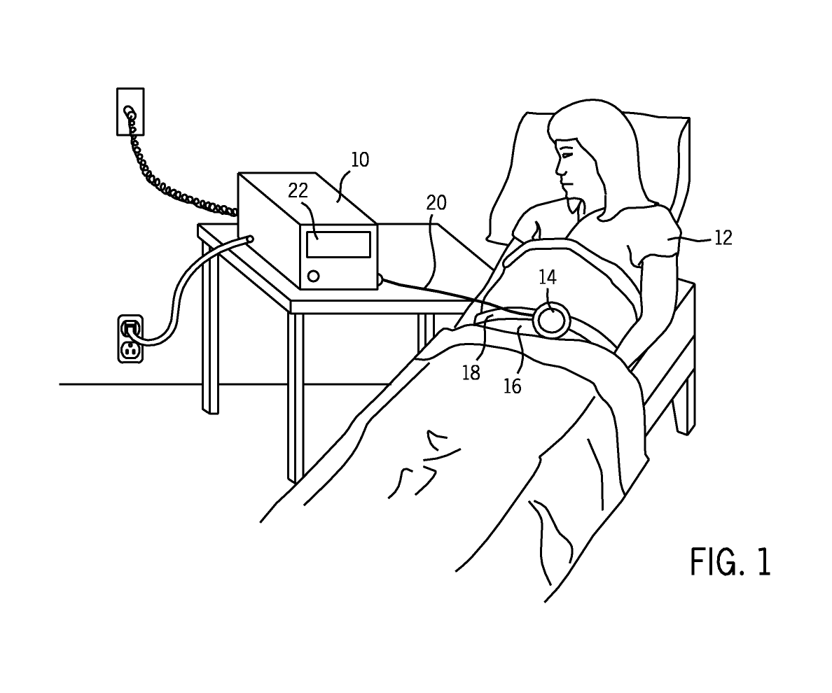

FIG. 1 illustrates a fetal heart rate monitor 10 that can be used to monitor the heart rate of a fetus of a pregnant patient 12. Although the fetal heart rate monitor 10 is shown in FIG. 1 in an exemplary form, it should be understood that the fetal heart rate monitor could take many other forms while operating within the scope of the present disclosure. One type of fetal heart rate monitor is the Corometrics 170 Series available from GE Healthcare.

In the embodiment of FIG. 1, the fetal heart rate monitor includes an ultrasound probe 14 that is secured to the patient's abdomen 16 by a strap 18. The ultrasound probe 14 is shown in the embodiment of FIG. 1 as being coupled to the fetal heart rate monitor 10 by a cable 20. However, in another possible embodiment, the fetal heart rate monitor 10 could communicate with the ultrasound probe 14 utilizing a wireless communication technique.

The fetal heart rate monitor 10 shown in FIG. 1 includes a display device 22, such as a screen or hard copy recording device, that typically displays the monitored heart rate of the fetus. The display screen 22 can be configured to display other monitored signals obtained from the patient in alternate embodiments.

During operation, when the fetal heart rate monitor 10 is powered on, one or more ultrasound transducers contained within the ultrasound probe 14 generate an ultrasound beam directed into the patient 12 through the skin of the abdomen. The fetal heart rate monitor monitors the ultrasound signals returned to either the same or different ultrasound transducers contained within the ultrasound probe to detect the beating of the fetal heart. Based upon data acquired from the ultrasound probe 14, the fetal heart rate monitor 10 calculates the fetal heart rate and displays the calculated fetal heart rate on the display 22 in a known manner.

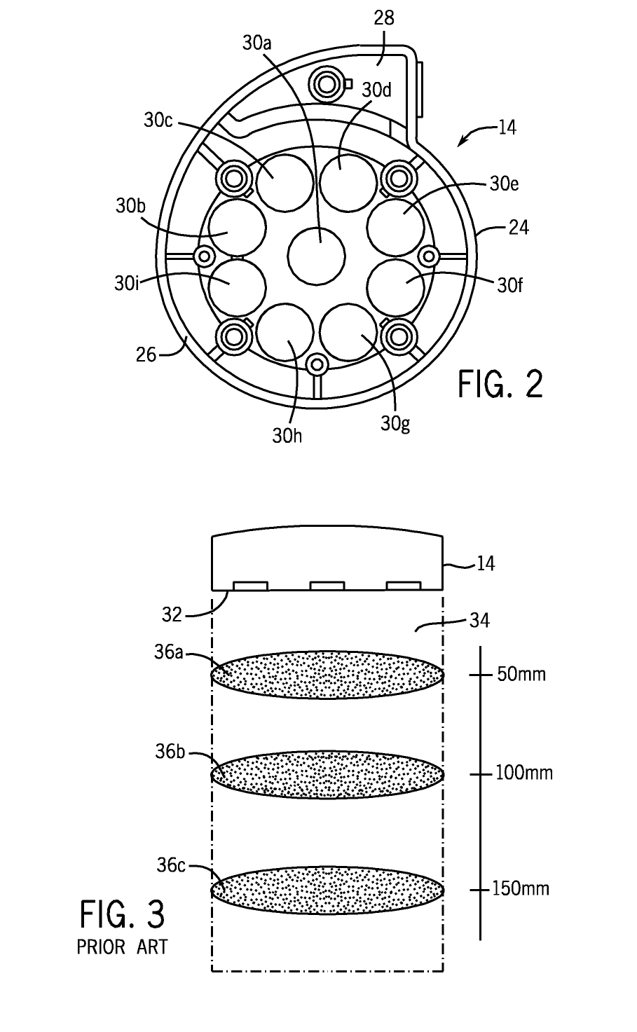

FIG. 2 illustrates a top view of the ultrasound probe 14 with the top half of the outer housing 24 removed. In the embodiment of FIG. 2, the outer housing 24 includes a generally circular outer wall having a protruding section 28 that allows for connection of the cable from the fetal heart rate monitor.

The ultrasound probe 14 shown in FIG. 2 includes a plurality of ultrasound transducers 30 positioned in a predetermined array. In the embodiment of FIG. 2, the ultrasound probe 14 includes nine individual transducers each independently operable. In the configuration of FIG. 2, a center transducer 30a is surrounded by eight outer transducers 30b-30i. Although nine ultrasound transducers are shown in FIG. 2, it should be understood that a different number of transducers could be utilized while operating within the scope of the present disclosure. The nine transducer array shown in FIG. 2 is a popular, available transducer arrangement such as is currently included in the Corometrics 170 Series monitor available from GE Healthcare.

During operation of the transducer probe 14 in accordance with known practices, each of the nine ultrasound transducers generates an ultrasound beam through a generally planar transmission surface 32, as shown in FIG. 3. The ultrasound beams from the transducers create a sensing region 34 that has approximately the same cross-sectional area at varying depths below the transmission surface 32. In the illustration of FIG. 3, the sensing area 36a at 50 mm is approximately the same as the sensing area 36b at 100 mm and the sensing area 36c at 150 mm. The interrogated volume is approximately cylindrical.

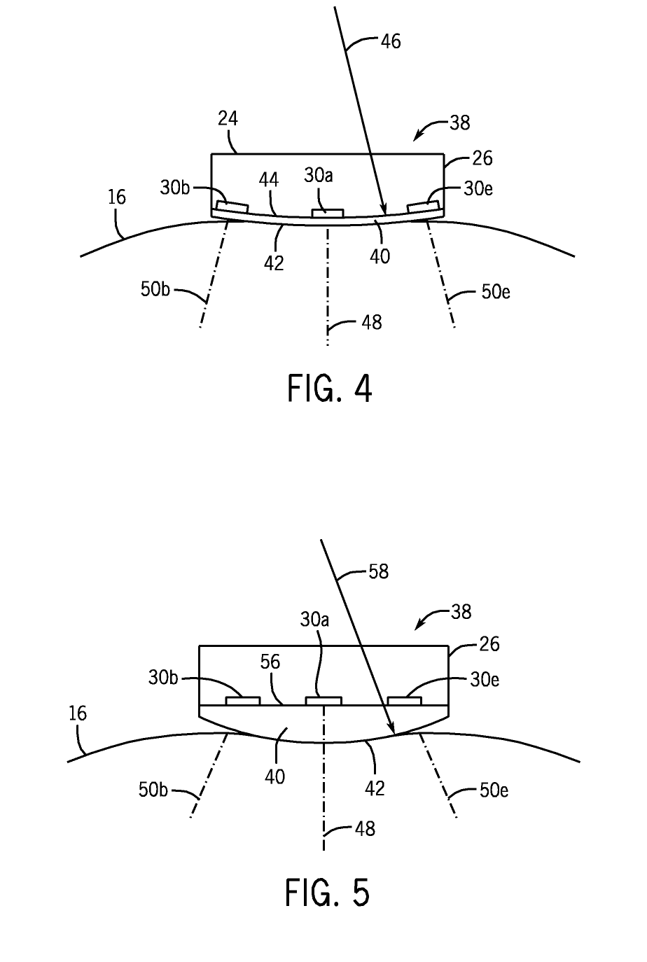

FIG. 4 illustrates a first embodiment of a defocused ultrasound probe generally referred to by reference character 38. The defocused ultrasound probe 38 includes the outer housing 24 including outer wall 26. In the embodiment shown, a transmission surface 40 is formed on the housing 24 and is curved such that the ultrasound transducers 30 are steered away from a center axis of the ultrasound probe. As illustrated in FIG. 4, the transmission surface 40 includes an outer face surface 42 and an inner face surface 44 spaced by the thickness of the transmission surface 40. When the ultrasound probe 38 is in use, the outer face surface 42 is positioned in contact with the patient's abdomen 16 such that the ultrasound beams from each of the ultrasound transducers 30 travel into the patient, as illustrated.

As shown in FIG. 4, the outer face surface 42 and the inner face surface 44 define the convex shape transmission surface 40 having an average radius of curvature (ROC) shown by line 46. The ROC can also be referred to as the focusing depth, which is behind the transmission surface 40 in the embodiment of FIG. 4. In the embodiment of FIG. 4, the focusing depth or radius of curvature is 300 mm, although other configurations are clearly contemplated as being within the scope of the present disclosure. In the embodiment of FIG. 4, the thickness of the transmission surface 40 is less than 1 mm.

As illustrated in FIG. 4, the center ultrasound transducer 30a generates an ultrasound beam transmitted along a center axis 48. Each of the outer ultrasound transducers 30b and 30e shown in FIG. 4 generate an ultrasound beam transmitted along a beam axis 50b and 50e, respectively. The curved shape of the transmission surface 40 causes the beam axis 50b, 50e of each of the outer ultrasound transducers 30b, 30e to diverge away from the center axis 48. For this reason, the probe 38 is referred to as a defocused probe.

Referring now to FIG. 5, thereshown is a second embodiment of a defocused ultrasound probe 38. In the second embodiment, the transmission surface 40 has an alternate configuration as compared to the embodiment of FIG. 4. In the embodiment of FIG. 5, the transmission surface 40 has a curved outer face surface 42 and a generally planar inner face surface 56. Each of the ultrasound transducers 30 are mounted to the generally planar inner face surface 56. In the embodiment of FIG. 5, the transmission surface 40 is an acoustic lens where the material is chosen such that the thickness causes each of the beam axes 50b, 50e to diverge from the center axis 48. In the embodiment of FIG. 5, the material used to form the transmission surface is a material with a sound velocity greater than the human body, such as Cycolac®, having an overall thickness of less than 2 mm. The transmission surface 40 also has a radius of curvature shown by line 58 though the acoustic focus depth may be very different depending on the speed of sound in the lens material. In the embodiment of FIG. 5, the curvature may be very slight such that the surface is nearly planar, but the acoustic focal depth may be similar to the more dramatically curved approach shown in FIG. 4.

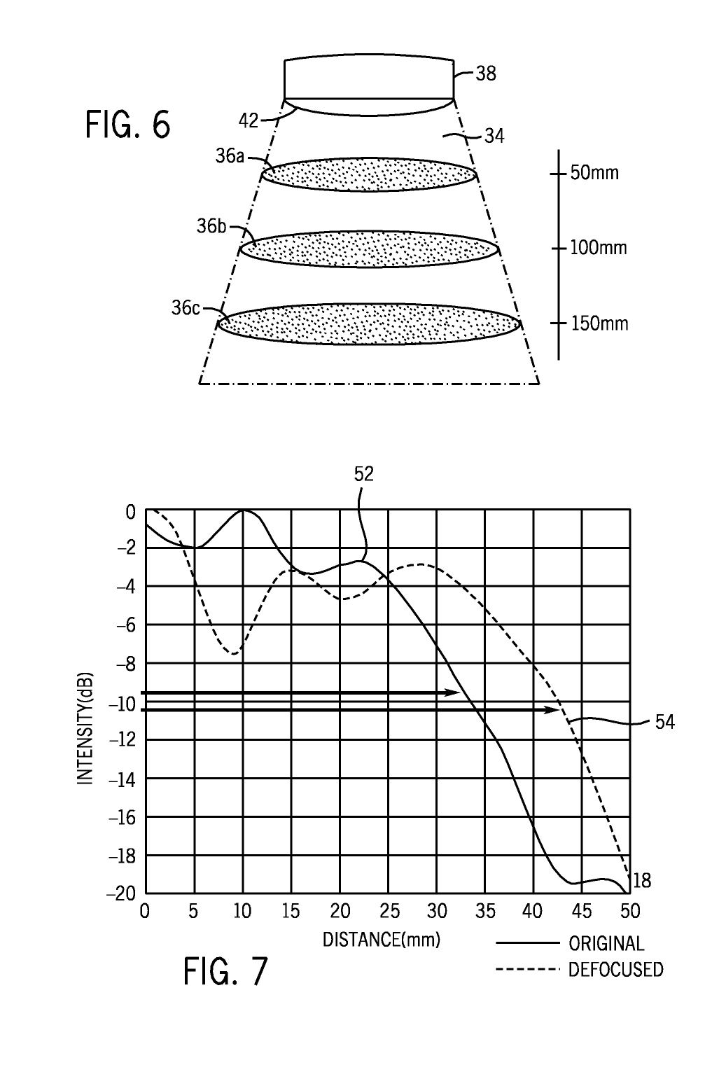

Referring now to FIG. 6, the defocused ultrasound probe 38 of FIG. 4 or 5 is illustrated having the convex outer face surface 42. Unlike the embodiment shown in FIG. 3, the sensing region 34 below the probe expands at increasing distances from the outer face surface 42. Specifically, the sensing area 36a at 50 mm from the outer face surface 42 is less than the sensing area 36b at 100 mm, which in turn is less than the sensing area 36c at 150 mm from the outer face surface 42. As can be clearly understood by comparison of FIGS. 3 and 6, the defocused ultrasound probe 38 expands the sensing volume while utilizing the same ultrasound transducer configuration.

FIG. 7 is a graphical depiction of the intensity of the combined ultrasound beam at varying distances from the center of the ultrasound probe at a depth of 150 mm from the outer face surface 42. Line 52 illustrates the intensity of the ultrasound beam utilizing the original, focused ultrasound probe 14 shown in FIG. 3. As line 52 indicates, at a distance of approximately 33 mm from the center of the probe, the signal intensity drops below −10 dB.

Line 54 illustrates that when the defocused ultrasound probe 38 of FIG. 4 is utilized, the intensity of the ultrasound beam drops below −10 dB at approximately 42 mm. Thus, the defocused ultrasound probe increases the effective radius from the centerline from approximately 33 mm to 42 mm, which results in a 62% increase in the area of coverage at 150 mm from the probe.

Although two embodiments for the transmission surface 40 are shown in FIGS. 4 and 5, it should be understood that various other configurations are contemplated as being within the scope of the present disclosure. The purpose of the transmission surface 40 is to cause the beam axis of each of the outer ultrasound transducers to diverge away from the center axis of the center ultrasound transducer. The diverging focus of the outer transducer elements creates an expanded sensing region, as was described in the comparison of FIGS. 3 and 6.

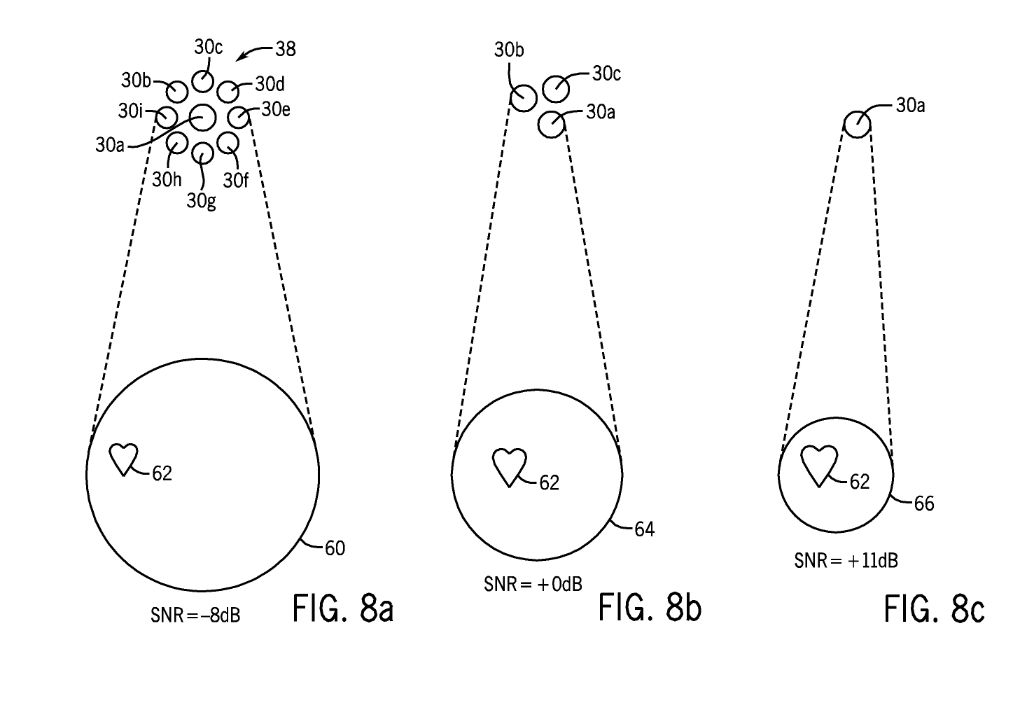

FIG. 8 demonstrates how the multiple transducers may be used in different combinations to increase or decrease the size of the ultrasound beam, or spotlight. Activating all transducers in parallel, FIG. 8a gives the largest sensing area; however, the best signal-to-noise may be achieved by activating just one transducer which is looking in the right direction. By choosing the correct single transducer beam or spotlight, fewer transducers are active, which reduces power and reduces the amount of energy transmitted into the body.

FIG. 8a illustrates the operation of the ultrasound transducers 30 within the defocused ultrasound probe 38. In the embodiment of FIG. 8a, all of the nine ultrasound transducers are simultaneously activated to create the ultrasound sensing area 60 having a beam width of 80 mm. As illustrated in FIG. 8a, the fetal heart 62 is contained within the sensing area 60 and thus can be detected by the defocused ultrasound probe 38. When all of the nine ultrasound transducers 30 are activate, the signal-to-noise ratio (SNR) is approximately −8 dB.

FIG. 8b illustrates the activation of only one combination of less than all of the plurality of ultrasound transducers. In the embodiment of FIG. 8b, three of the nine ultrasound transducers, namely center transducer 30a and outer transducers 30b and 30c, are simultaneously activated. When the combination shown in FIG. 8b is activated, a smaller, more focused sensing area 64 is created, which still includes the fetal heart 62. In the embodiment illustrated, the sensing area 64 has a beam width of approximately 50 mm, which is less than the 80 mm beam width of the sensing area 60 of FIG. 8a. As illustrated in FIG. 8b, the signal-to-noise ratio of the sensing area 64 is 0 dB, which is an improvement from the SNR when all nine of the ultrasound transducers are activated, as illustrated in FIG. 8a. Although the sensing area 64 is reduced relative to the sensing area 60, the improved SNR illustrates that the use of only a select number of the ultrasound transducers is an improvement over the use of all of the ultrasound transducers, assuming the fetal heart 62 is within the sensing area 64.

FIG. 8c illustrates yet another alternate method in which only a single ultrasound transducer is activated. In the embodiment of FIG. 8c, one of the outer transducers 30b is activated. However, it should be understood that any one of the nine ultrasound transducers could be utilized, and that each transducer may produce beams that acoustically sense different locations in the body.

The sensing area 66 created by the single ultrasound transducer is dramatically smaller than the sensing area 60 created by all nine transducers. In the embodiment of FIG. 8c, the width of the sensing area 66 is approximately 30 mm. The sensing area 66 shown in FIG. 8c includes the fetal heart 62 and has a dramatically improved signal-to-noise ratio of approximately +11 dB. Although the single ultrasound transducer shown in FIG. 8c provides improved signal-to-noise ratio, the size of the sensing area 66 is significantly reduced relative to the use of either nine or three transducers.

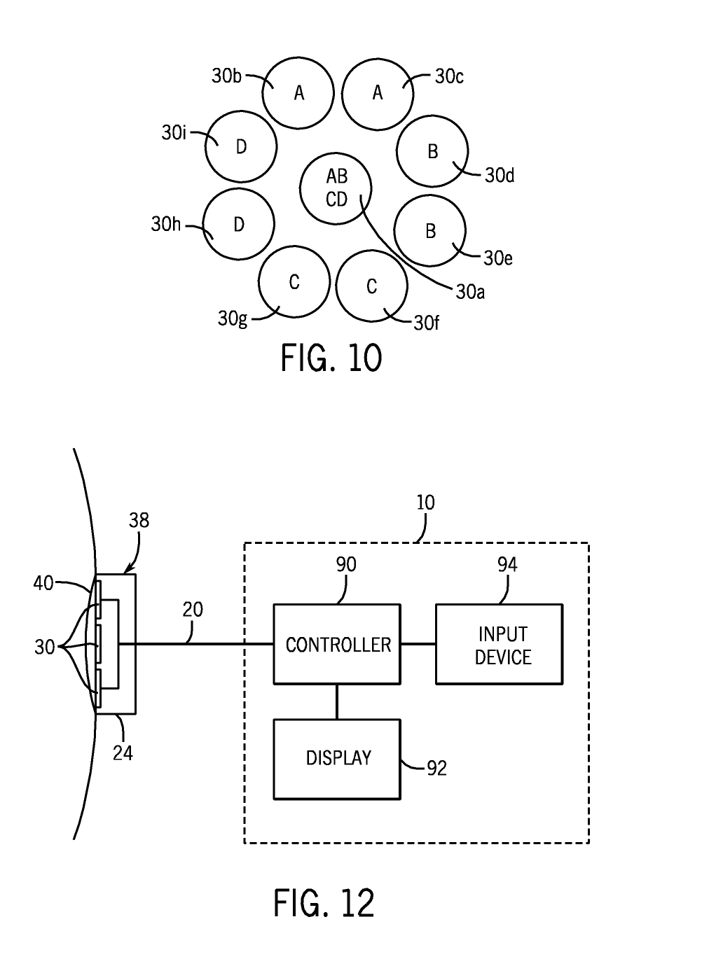

FIG. 10 graphically depicts one example of multiple combinations of the nine transducers that can be activated to create the sensing area 64 of FIG. 8b. In each combination, three transducers are simultaneously activated. In an embodiment that includes a defocused probe, the transmission surface causes the ultrasound beams from each of the outer transducers 30b-30i to diverge from the center axis created by the ultrasound beam of the center ultrasound transducer 30a. In the embodiment of FIG. 10, four separate combinations of the ultrasound transducers are proposed. The first combination (A) includes the center transducer 30a and outer transducers 30b and 30c. The second combination (B) includes the center transducer 30a and the outer transducers 30d and 30e. The third combination (C) includes the center transducer 30a and the outer transducers 30f and 30g. The fourth and final combination (D) includes the center transducer 30a and the outer transducers 30h and 30i. Although four proposed combinations are shown in FIG. 10, it is contemplated that additional combinations could be proposed and utilized while operating within the scope of the present disclosure.

In addition to combining several transducers to create a combination, it is also contemplated that each of the individual transducers could be operated individually as one of the proposed combinations.

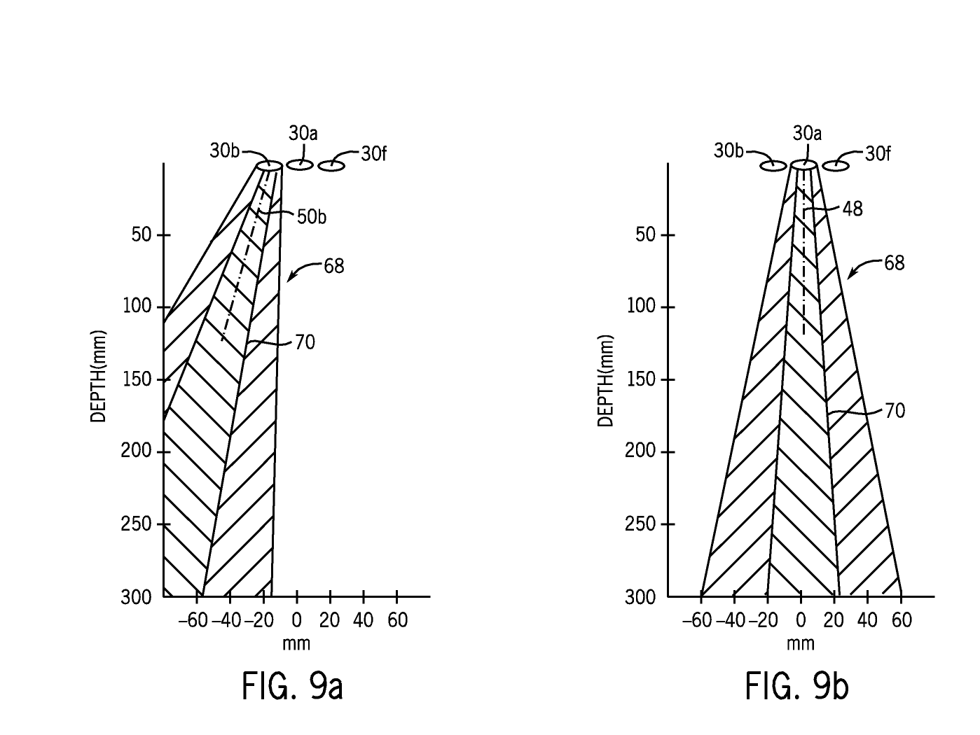

FIG. 9a illustrates the operation of a single transducer which creates a “spotlight” effect to look for the fetal heart beat. In the embodiment of FIG. 9a, the outer transducer 30b is operated to create the ultrasound beam 68. In the embodiment illustrated, the ultrasound beam 68 is centered along the beam axis 50b. As illustrated in FIG. 9a, at a depth of 150 mm, the center section 70 of the ultrasound beam 68 covers from approximately −30 mm to approximately −65 mm from the center axis of the housing. The center sections 70 indicate the optimal sensitivity for this beam, or −6 dB signal. The outer profile is a −10 dB indicator for the beam, which is wider, but signal sensitivity is marginally good. The defocusing probe may be designed such that the −10 dB profile of the beams from different transducer elements 30a and 30b overlap such that the entire volume is sufficiently sampled by the available elements.

Alternatively, the center ultrasound transducer 30a may be activated alone, as shown in FIG. 9b. The ultrasound beam 68 from the center transducer 30a extends along the center axis 48. At the same depth of 150 mm, the center section 70 covers from approximately −20 to +20 mm from the center axis of the ultrasound probe. As can be understood in FIGS. 9a and 9b, the sequential operation of each one of the individual ultrasound transducers allows the ultrasound probe to selectively spotlight different areas beneath the ultrasound probe. As described in FIG. 8c, although the sensing area 66 is reduced relative to the activation of all nine transducers simultaneously, the increased signal-to-noise ratio provides benefits as have been previously described.

The system may choose to activate any of the transducers in a pattern to search for the best heart rate signal.

As can be understood in the embodiments of FIGS. 9a and 9b, since each of the transducers creates an ultrasound beam that diverge from each other due to the defocusing transmission surface, it is contemplated that the ultrasound probe could be utilized to monitor two fetal heart beats, such as when the patient is pregnant with twins. In such an embodiment, one or more of the ultrasound transducers would request the first fetal heart beat while a second combination of the multiple transducers could detect the second fetal heart beat. Once the two heart beats are detected, the system would continue to monitor the separate heart beats utilizing different combinations of the ultrasound transducer.

Although the embodiment shown in FIGS. 8-10 is described as utilizing a defocused ultrasound probe 38 including a transmission surface, it should be understood that the same method could be utilized with a standard ultrasound probe having an essentially flat transmission surface.

Referring now to FIG. 12, thereshown is a schematic illustration of the operating components of the fetal heart rate monitor 10. The fetal heart rate monitor 10 includes a controller 90 that operates to control the operation of the patient monitor, which may include the selecting of transducers to activate, generation of ultrasound excitation signals, and Doppler processing of the received signals. The controller 90 is shown connected to the defocused ultrasound probe 38 through cable 20. Through the cable 20, the controller 90 can control the selective operation of the transducers 30 contained within the probe housing 24. As previously described, each of the transducers 30 are positioned behind the transmission surface 40.

The controller 90 is further connected to a display 92 for visually indicating to the operator the detected fetal heart rate. The controller can preferably also include an input device 94 that allows the operator to input information into the controller as desired.

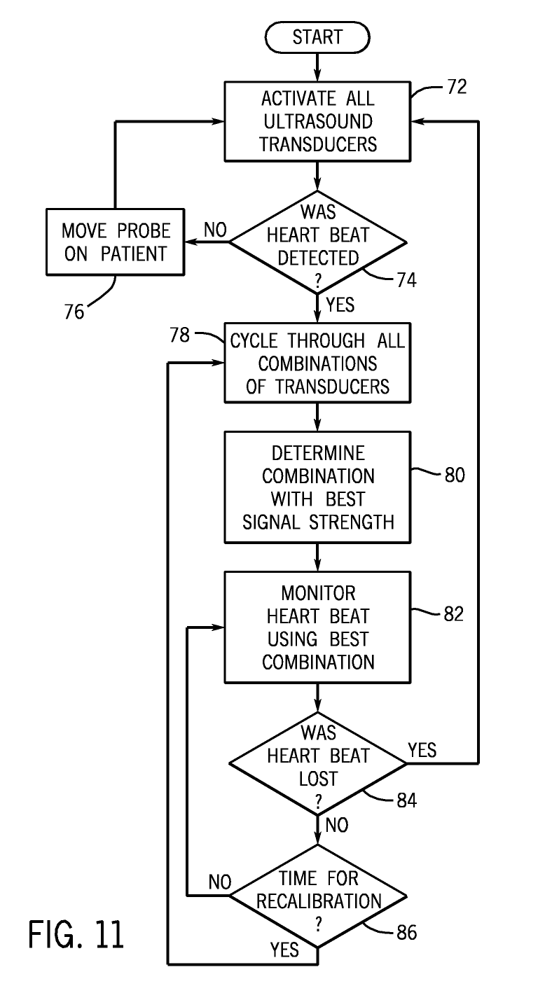

FIG. 11 illustrates one proposed method of operating the ultrasound probe including the plurality of ultrasound transducers, such as the nine ultrasound transducers shown in FIG. 10.

Initially, a clinician may place the defocused ultrasound probe having the transmission surface on the abdomen of a patient. Once the ultrasound probe is positioned, the controller activates all nine of the ultrasound transducers, as illustrated in step 72. In the ultrasound probe shown in FIG. 10, the controller activates all nine of the ultrasound probes to create the broadest sensing area possible from the ultrasound probe, as shown in FIG. 8a.

The controller determines at step 74 whether the heart beat was detected from the fetus. The step of determining whether the heart beat was detected may require multiple cycles before the controller determines whether the heart beat was detected. If the heart beat was not detected in step 74, the operator will move the probe, as illustrated in step 76, and attempt to locate the fetal heart signal.

Once the controller determines that the probe is in position such that at least one of the ultrasound transducers detects the fetal heart rate, the operator may signal to the controller to search for the optimal transducer to track the heart rate. The controller activates all of the combinations of transducers in sequential order, as illustrated in step 78. As described with respect to FIG. 10, four possible combinations (A-D) of three transducers each are proposed for the nine transducer configuration of FIG. 10. Alternatively, each combination could include only one of the nine transducers or other combinations could be created as desired.

As the controller activates each of the combinations of the nine transducers, the controller determines whether each combination detects the fetal heart rate and also the relative signal strength of the heart rate. Since each combination of transducers creates a combined ultrasound beam having a different directional component due to the defocusing transmission surface, it is contemplated that a few of the combinations will detect the fetal heart rate.

Once the controller has cycled through all of the combinations of the transducers, the controller determines in step 80 which of the combinations resulted in the best heart rate signal strength. Typically, this comparison is conducted using proprietary or established signal analysis methods.

Once the controller selects the combination of transducers with the best signal strength in step 80, the controller operates the fetal heart rate monitor to continuously sense the fetal heart rate using the selected best combination, as illustrated in step 82. Since the combination proposed includes three transducers, the sensing area and signal-to-noise ratio will be approximated by the illustration of FIG. 8b. The use of a cycling method between the multiple combinations allows the controller to first locate which combination best detects the fetal heart rate and, once the best combination has been determined, continue to monitor the fetal heart rate using only the selected combination. This method increases the effective overall coverage of the ultrasound probe while increasing the signal-to-noise ratio during continuous monitoring.

During monitoring of the fetal heart rate, the controller determines whether the heart beat is ever lost, as illustrated in step 84. Since the fetus is moving within the pregnant patient, the position of the fetus can change, which may result in a loss of the heart beat signal. If the controller determines in step 84 that the heart beat is lost, the controller returns to step 72 and activates all of the ultrasound transducers to determine whether the fetal heart rate is still in a sensing position beneath the ultrasound probe. If the heart beat is again detected in step 74, the method continues as described. However, if the fetus has moved a significant amount and can no longer be sensed by the ultrasound probe, the system signals to the attendant that the probe must be moved, as illustrated in step 76.

If the controller determines in step 84 that the heart beat was not lost, the system may again cycle through different transducer combinations to determine the optimal sensor configuration, beginning again with step 78. These recalibration steps may be performed even when the fetal heart signal is not lost, to maintain constant and consistent optimal tracking of the fetal heart rate. Thus, if the system determines in step 86 that it is time for a new recalibration, the system returns to step 78 and activates all of the combinations of the transducers. It is contemplated that recalibration can occur at a regular interval, such as every five, ten or fifteen minutes.

The controller continues to follow the flowchart of FIG. 11 and monitors the fetal heart rate as desired.

In an alternate embodiment, the system can activate only the center ultrasound transducer in step 72 instead of activating all of the nine ultrasound transducers. The use of only the center transducer will allow an operator to position the ultrasound probe such that the center transducer is able to detect the fetal heart rate. When the center transducer can detect the fetal heart rate, the ultrasound probe is best centered around the fetus such that should the fetus move slightly, one or more of the outer transducers will most likely be able to detect the fetus. Having the probe optimally centered initially will allow for greater movement range by the fetus without the need for the operator to reposition the probe.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

We claim:

1. An ultrasound probe for use with a heart rate monitor, the ultrasound probe comprising:
an outer housing;
a transmission surface coupled to the housing; and
a plurality of ultrasound transducers contained within the outer housing, each ultrasound transducer positioned to generate an ultrasound beam through the transmission surface,
wherein the transmission surface is shaped to cause the ultrasound beams from the plurality of ultrasound transducers to diverge from each other.

2. The ultrasound probe of claim 1 wherein the transmission surface includes a convex outer face surface and a convex inner face surface.

3. The ultrasound probe of claim 2 wherein the plurality of ultrasound transducers are each mounted to the convex inner face surface.

4. The ultrasound probe of claim 1 wherein the plurality of ultrasound transducers include at least a center transducer and a plurality of outer transducers, wherein the center transducer generates a center ultrasound beam along a center axis and the ultrasound beams from each of the plurality of outer transducers diverge from the center axis.

5. The ultrasound probe of claim 1 wherein the transmission surface includes a convex outer face surface and a generally planar inner face surface.

6. The ultrasound probe of claim 5 wherein the plurality of ultrasound transducers are each mounted to the inner face surface.

7. The ultrasound probe of claim 5 wherein the transmission surface is formed as part of the outer housing.

8. A method of operating a heart rate monitor having an ultrasound probe including a plurality of ultrasound transducers each operable to generate an ultrasound beam, the method comprising the steps of:
creating a plurality of combinations of the ultrasound transducers, wherein each combination includes less than all of the plurality of ultrasound transducers;
activating one or more of the combinations of the plurality of ultrasound transducers to detect a heart beat;
determining which combination of the plurality of ultrasound transducers detects the heart beat; and
operating only the determined combination of the plurality of ultrasound transducers to monitor the heart rate.

9. The method of claim 8 wherein the ultrasound probe includes nine ultrasound transducers and each combination includes three of the ultrasound transducers.

10. The method of claim 9 wherein the nine ultrasound transducers include a center ultrasound transducer and eight outer ultrasound transducers, wherein each combination includes the center ultrasound transducer and two of the outer transducers.

11. The method of claim 8 wherein each combination includes only one ultrasound transducer.

12. The method of claim 8 further comprising the steps of:
monitoring for the loss of the detected heart beat during operation of the determined combination;
upon loss of the heart beat, reactivating one or more combinations of the plurality of ultrasound transducers to detect the heart beat; and
determining which combination of the plurality of ultrasound transducers detects the heart beat signal.

13. The method of claim 8 further comprising the steps of:
determining if more than one combination of the plurality of ultrasound transducers detects a heart beat;
selecting the combination of ultrasound transducers that produces the best signal for the heart beat; and
operating only the selected combination of the ultrasound transducers to monitor the heart rate.

14. The method of claim 8 further comprising the steps of:
operating all of the plurality of ultrasound transducers to detect the heart beat beneath the ultrasound probe;
moving the ultrasound probe on the patient until a heart beat is detected;
activating one or more of the combinations of the plurality of ultrasound transducers;
determining which of the activated combinations of the plurality of ultrasound transducers best detects the heart beat; and
operating only the determined combination of the plurality of ultrasound transducers to monitor the heart rate.

15. A method of operating a fetal heart rate monitor having an ultrasound probe including a plurality of ultrasound transducers contained within an outer housing, each ultrasound transducer being operable to generate an ultrasound beam from the housing, the method comprising:
transmitting an ultrasound beam from each of the ultrasound transducers through a transmission surface, the transmission surface being formed to cause the ultrasound beams of the plurality of ultrasound transducers to diverge from each other;
creating a plurality of combinations of the ultrasound transducers, wherein each combination includes less than all of the plurality of ultrasound transducers;
activating each combination of the plurality of ultrasound transducers;
determining which combination of the plurality of ultrasound transducers detects a heart beat; and
operating only the determined combination of the plurality of ultrasound transducers to monitor the heart rate.

16. The method of claim 15 wherein the ultrasound probe includes nine ultrasound transducers and each combination includes three of the ultrasound transducers.

17. The method of claim 16 wherein the nine ultrasound transducers include a center ultrasound transducer and eight outer ultrasound transducers, wherein each combination includes the center ultrasound transducer and two of the outer transducers.

18. The method of claim 15 wherein each combination includes only one ultrasound transducer.

19. The method of claim 15 further comprising the steps of:
monitoring for the loss of the detected heart beat during operation of the determined combination;
upon loss of the heart beat, reactivating one or more combinations of the plurality of ultrasound transducers to detect the heart beat; and
determining which combination of the plurality of ultrasound transducers detects the heart beat.

20. The method of claim 15 further comprising the steps of:
determining if more than one combination of the plurality of ultrasound transducers detects a heart beat;
selecting the combination of ultrasound transducers that produces the best signal for the heart beat; and
operating only the selected combination of the ultrasound transducers to monitor the heart rate.

21. The method of claim 15 further comprising the steps of:
operating all of the plurality of ultrasound transducers to detect the heart beat beneath the ultrasound probe;
moving the ultrasound probe on the patient until a heart beat is detected;
activating each combination of the plurality of ultrasound transducers;
determining which combination of the plurality of ultrasound transducers best detects the heart beat; and
operating only the determined combination of the plurality of ultrasound transducers to monitor the heart rate.
